(19) United States
(12) Patent Application Publication
(10) Pub. No.: US 2026/0354723 A1
(43) Pub. Date: Nov. 12, 2026

(54) TRANSDERMAL MEDICAL DEVICE AND KIT COMPRISING THE SAME

(71) Applicant: Roche Diagnostics Operations, Inc., Indianapolis, IN (US)

(72) Inventors: Ute Baer, Mannheim (DE); Alina Gottselig, Mannheim (DE); Florian Grupp, Mannheim (DE); Hans Zahn, Mannheim (DE)

(21) Appl. No.: 19/706,823

(22) Filed: Jun. 12, 2026

(30) Foreign Application Priority Data
Dec. 14, 2023 (EP) 23216782.5
Dec. 13, 2024 (WO) PCTEP2024086309

(51) Int. Cl. A61B 5/151; A61B 5/15

(52) U.S. Cl. CPC A61B 5/15194; A61B 5/150022; A61B 5/150175; A61B 5/150503; A61B 5/15117; A61B 5/15123; A61B 5/1513

(57) ABSTRACT

A transdermal medical device, specifically for sampling of capillary blood, comprises at least one stationary component and at least one movable component mounted to the stationary component in a movable manner. The movable component is movable from a distal position, in which at least one penetration element of the movable component is received within the stationary component, to a proximal position, in which the penetration element protrudes from an application side of the stationary component for penetrating the skin of a user. The movable component further comprises at least one magnetic driver element configured such that a movement of the movable component is drivable by at least one external magnetic force. A body mount for attaching the transdermal medical device to a body surface of the user, a transdermal medical kit comprising the transdermal medical device and the body mount, and a method of driving a penetration element are proposed.

### CROSS-REFERENCE TO RELATED APPLICATIONS

This is a continuation of and claims priority to European Application No. 23216782.5 filed on Dec. 14, 2023 and entitled “TRANSDERMAL MEDICAL DEVICE AND KIT COMPRISING THE SAME,” which claims priority to International Application No. PCT/EP/086309 filed on Dec. 13, 2024 and entitled “TRANSDERMAL MEDICAL DEVICE AND KIT COMPRISING THE SAME,” the entire contents of which are incorporated herein by reference.

### TECHNICAL FIELD

The invention relates to a transdermal medical device, to a body mount, to a transdermal medical kit comprising the transdermal medical device and the body mount, and to a method of driving a penetration element. The devices and the method according to the invention specifically may be used in the field of diagnostic sampling, both in homecare and in a professional medical environment. Specifically, the devices and the method may be used for sampling of capillary blood. The devices and methods, however, may also be used for other purposes requiring penetration of the skin of a user, such as for injection purposes, e.g. for injecting one or more liquid medicaments into a body tissue and/or into blood vessels. Other applications of the present invention are also feasible.

### BACKGROUND ART

Both in the field of home care and in professional medical environments such as hospitals or nursing homes, means and methods for penetrating the skin of a user are widely used. In the following, the invention specifically will be described in the context of sampling of capillary blood. It shall be noted, however, that other applications, such as applications for injecting medically active components into the body tissue and/or into blood vessels of the user, are also feasible.

For penetrating the skin of the user, besides traditional manual means using manually-handled lancets or needles, automatic devices are known and becoming more and more popular. Specifically, means and methods for diagnostic sampling of capillary blood are applied e.g. in the field of self-sampling or home sampling. These means and methods require a penetration of the skin and, specifically, a punctuation of capillary blood vessels. Subsequently, samples of blood, such as droplets of capillary blood, are collected by using appropriate collection means, such as paper, plasma separation cards or sample tubes. The samples may then be analyzed, either at home or by shipping these samples to analytical facilities. Home sampling, meanwhile, is widely used, specifically for avoiding frequent visits to the doctor or to the hospital.

The penetration of the own skin for self-sampling typically takes some effort for the user. Automatic devices avoiding the necessity of manually pricking e.g. the fingertip with a needle or a lancet are rather helpful for overcoming these efforts. Devices for punctuating the skin are commercially available, such as by Tasso, Inc., Seattle, WA 98119, USA, or by YourBio Health, Inc., Medford, MA 02155, USA. The triggering of these sampling devices may either take place by a pushbutton to be actively pushed by the user or by pressing the device onto the skin, whereby the device is triggered and a needle or lancet, driven by a spring, penetrates the skin.

US 2008/228212 A1 relates to lancet device by which a lancet is displaceable along a piercing path to generate a piercing wound in a skin surface, in particular to obtain body fluid for diagnostic purposes. To achieve performance of a piercing movement which is low in noise and shock, the lancet drive may include a control unit for regulating the piercing depth of the lancet and the lancet may be implemented to oscillate freely out in the piercing direction without delimitation by a mechanical piercing depth stop.

US 2005/0125017 A1 describes a lancet device (10) having an internal magnet (20) and the forces emanating therefrom driving and retracting a collar (30) and a lancet (60). The device has a steady state condition (SS) wherein the device is at rest and in equilibrium. The device can be armed and activated to puncture. Once puncturing has occurred the device returns to a steady state condition.

WO 2019/226754A1 proposes a device (1) for obtaining a biological sample that includes a blood collection device (10) and a vibration device (80) removably attachable to the blood collection device that provides pain relief. The vibration device of the present disclosure provides pain relief to a patient by vibrating the blood collection device. The vibration device can be attached to the blood collection device before it is placed onto a finger to provide pain relief during the finger lancing as well as to aid blood flow from the finger into a collection container during the collection process.

U.S. Pat. No. 6,925,317 B1 describes an alignment device and related systems and methods for aligning at least one apparatus with respect to a surface of a tissue. The alignment device comprises a tissue interface member suitable for positioning on the surface of the tissue and mating with the apparatus to maintain alignment of the apparatus during an operation of the apparatus. The alignment device is useful to align various apparatus that are part of a continuous analyte monitoring system.

One technical challenge, however, which is typically present in these devices for blood sampling, resides in the control of the punctuation depth. In case the punctuation depth is too low, an insufficient amount of blood is generated. A high punctuation depth, contrarily, increases the discomfort for the user and may lead to an excess amount of blood and a contamination of the user's personal belongings and the like. These challenges are increased by the fact that, by pressing the device onto the body surface, the body tissue is compressed, so the penetration depth is typically increased by the device itself. Even worse, the penetration depth may vary, depending on the properties of the body tissue and/or depending on the pressure exerted with the device onto the body surface.

Consequently, specifically in the field of self-sampling or home sampling, but also in other areas in which transcutaneous devices are applied, there is a need for devices which generate a reliable and reproducible penetration depth, which, specifically, should be widely independent on the behavior of the user and of the handling of the device. The penetration created by the device should specifically be sufficient for sampling, but should not be excessive, in order to avoid contaminations of the user's personal belongings by excess blood. Further, the process of penetration and, specifically, the process of collecting the sample should be convenient and straightforward.

### Problem to Be Solved

It is therefore desirable to provide a transdermal medical device, a body mount, a transdermal medical kit, and a method of driving a penetration element, which at least partially address the above-mentioned technical challenges of known devices and methods. The devices and method specifically should be applicable in the field of blood sampling and/or in the field of injection of medically active components. The devices and method specifically should provide a high reliability regarding the penetration depth, the amount of bodily fluid should be reproducible, and the use of the devices should be convenient and straightforward.

### SUMMARY

This problem is addressed by a transdermal medical device, by a body mount, by a transdermal medical kit, and by a method of driving a penetration element, with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, a transdermal medical device is proposed. The transdermal medical device comprises at least one stationary component and at least one movable component. The movable component is mounted to the stationary component in a movable manner. The movable component is movable from a distal position to a proximal position. The movable component comprises at least one penetration element configured for penetrating the skin of a user. The penetration element, in the distal position of the movable component, is received within the stationary component. In the proximal position of the movable component, the penetration element protrudes from an application side of the stationary component. The movable component further comprises at least one magnetic driver element which is configured such that a movement of the movable component is drivable by at least one external magnetic force.

The term “transdermal”, as used herein, is a broad term and is to be given its ordinary and customary meaning to a person of ordinary skill in the art and is not to be limited to a special or customized meaning. The term specifically may refer, without limitation, to the property of a process of taking place through the skin of a user. Thus, a process described as “transdermal” typically comprises a penetration of the skin, either by directly creating the penetration or by using a penetration already present. In the following, the term specifically will be used for indicating that a device is configured for penetrating the skin or that a process implies the penetration of the skin.

The term “medical device”, as used herein, is a broad term and is to be given its ordinary and customary meaning to a person of ordinary skill in the art and is not to be limited to a special or customized meaning. The term specifically may refer, without limitation, to an arbitrary device having one or more components, the device being configured for performing one or more medical actions or contributing to one or more medical actions, such as one or more of a diagnostic action and a therapeutic action.

The term “stationary component”, as used herein, is a broad term and is to be given its ordinary and customary meaning to a person of ordinary skill in the art and is not to be limited to a special or customized meaning. The term specifically may refer, without limitation, to a component of a device, the component having one or more elements, the component being, in the intended use, widely stationary with respect to a chosen inertial system or coordinate system, such as the coordinate system of at least one of: a coordinate system of the user's hand holding the transdermal medical device, a coordinate system of the transdermal medical device itself, a coordinate system of the body surface of the user. The term “stationary component” specifically may also be used to distinguish said component from one or more movable parts, such as the movable component described in further detail below.

Similarly, the term “movable component”, as used herein, is a broad term and is to be given its ordinary and customary meaning to a person of ordinary skill in the art and is not to be limited to a special or customized meaning. The term specifically may refer, without limitation, to a component of a device, the component having one or more elements, the component being, in the intended use, movable within a chosen inertial system or coordinate system, such as the coordinate system of at least one of: a coordinate system of the user's hand holding the transdermal medical device, a coordinate system of the transdermal medical device itself, a coordinate system of the body surface of the user. The term “movable component” specifically may also be used to distinguish said component from one or more stationary parts, such as the stationary component described above. Consequently, the terms “stationary component” and “movable component” specifically may be used as mere nomenclature, indicating that the stationary component may define a coordinate system in which the movable component is movable, in one or more linear movements and/or in one or more nonlinear movements and/or in one or more rotational movements.

As outlined above, the movable component is mounted to the stationary component in a movable manner, such that the movable component is movable from a distal position to a proximal position. Specifically, the movement may be a linear movement. Other movements, however, are also feasible. The stationary component may also provide for one or more guiding elements, such as one or more guiding surfaces, configured for guiding the movement of the movable component from the distal position to the proximal position. The movement specifically, as will be outlined in further detail below, may also be a reversible movement, such that the movable component may move back, from the proximal position, to the distal position. Therein, the direction of movement from the distal position to the proximal position may define a penetration direction, wherein the opposite direction of movement, i.e. the movement from the proximal position to the distal position, may define a retraction direction of the movable component.

The terms “distal position” and “proximal position”, as used herein, are broad terms and are to be given their ordinary and customary meaning to a person of ordinary skill in the art and are not to be limited to a special or customized meaning. The terms specifically may be used as mere nomenclature to distinguish the positions of the movable component. The terms specifically, however, may be used in the context of the intended use of the transdermal medical device. Thus, in the intended use, the distal position may be a position of the movable component in which the movable component is located farthest away from the skin of the user, whereas the proximal position may be a position of the movable component in which the movable component is located closest to the skin of the user. Specifically, the proximal position may be a position in which the penetration element may penetrate the skin of the user when the transdermal medical device is applied to the body mount and the body mount is applied to the skin of the user, as will be outlined in further detail below. The distal position on the other hand may be a position in which the penetration element may be located entirely outside the skin of the user when the transdermal medical device is applied to the body mount and the body mount is applied to the skin of the user.

The term “application side”, as used herein, is a broad term and is to be given its ordinary and customary meaning to a person of ordinary skill in the art and is not to be limited to a special or customized meaning. The term specifically may refer, without limitation, to a side of the stationary component which, in the intended use, faces the body surface of the user. The application side specifically may comprise at least one application surface which may directly or indirectly be applied to the body surface of the user. Thus, the application surface may directly rest on the body surface, or the application surface may rest on at least one intermediate component interposed in between the body surface and the stationary component, such as the body mount as described in further detail below. The application surface on the application side specifically may be or may comprise a ring-shaped surface, such as the ring-shaped surface of a sleeve forming the stationary component or a part thereof. The application side, specifically the application surface on the application side, specifically may be a part of the stationary component from which, during penetration, the penetration element protrudes.

The transdermal medical device, on the application side, may further comprise at least one sealing element and/or protection element. Thus, as an example, a sterile seal may be provided. As an example, the application side may fully or partially be covered with at least one protective liner which may be removed by the user before use of the transdermal medical device, specifically before applying the transdermal medical device to the body mount.

The term “penetration element”, as used herein, also often referred to as a “needle”, is a broad term and is to be given its ordinary and customary meaning to a person of ordinary skill in the art and is not to be limited to a special or customized meaning. The term specifically may refer, without limitation, to an arbitrary element configured for penetrating the skin and optionally also the body tissue of the user. For this purpose, the penetration element specifically may comprise at least one of a sharp and a point. More specifically, the penetration element may comprise at least one of a needle and a cannula. The penetration element may have an elongated shape, with the sharp or point being located at a proximal end of the penetration element. The penetration element may fully or partially be made of a solid material or element or may also be made as a hollow or slotted element. The penetration element, as an example, may be made of at least one of a metallic material and a plastic material. Other materials, however, are also feasible.

The movable component may comprise a movable body, such as a movable body made as a solid cylinder or block or a hollow movable body, such as a movable shell, having one or more movable inner spaces, with the penetration element mounted thereto, such as with the penetration element protruding, on a side facing the application side, from the movable body. The movable body may comprise one or more components, such as a movable upper part and a movable lower part, which may be joined by one or more connection elements during assembly. The at least one movable inner space may be located in between the movable upper part and the movable lower part, and one or more further components may be received therein, such as the magnetic driver element and/or a needle holder holding the penetration element. As an example, the movable body may be made of a plastic material, whereas the penetration element, as outlined above, may be made e.g. of a metallic material. The movable body of the movable component specifically may be made by molding techniques, such as injection molding, and the penetration element may be integrated therein or mounted thereto e.g. by material connection, such as by insert molding. Other techniques, however, are also feasible.

As further outlined above, the penetration element, in the distal position of the movable component, is received within the stationary component and, in the proximal position of the movable component, protrudes from the application side of the stationary component. Thus, in the distal position, the stationary component may act as a housing which fully or partially surrounds the penetration element and possibly further parts of the movable component, such as at least a part of the above-mentioned movable body of the movable component. In the proximal position of the movable component, however, the penetration element specifically may protrude from the application side of the stationary component. Specifically, the stationary component may provide for a housing such as a sleeve in which the movable component may move, specifically slide, from the distal position to the proximal position, wherein, in the distal position, the penetration element is fully circumferentially surrounded by the sleeve formed by the stationary component and wherein, in the proximal position, the penetration element protrudes from the application side, specifically from the sleeve, more specifically from an opening surrounded by a rim of the sleeve.

The term “magnetic driver element”, as used herein, is a broad term and is to be given its ordinary and customary meaning to a person of ordinary skill in the art and is not to be limited to a special or customized meaning. The term specifically may refer, without limitation, to magnetic element being part of at least one driving mechanism for driving at least one component of a system or a device. The magnetic driver element specifically may comprise at least one magnetic material, such as at least one permanent magnet. The magnetic material may be formed or may form a permanent magnet or, alternatively, may also be integrated into one or more other components, such as by the magnetic driver element comprising magnetic particles comprised as a filler material in a plastic material. Various alternatives are feasible. The magnetic driver element is integrated or attached to the movable component, such that a movement of the movable component may be controlled by at least one external magnetic force. The term “external magnetic force”, as used herein, also is a broad term and is to be given its ordinary and customary meaning to a person of ordinary skill in the art and is not to be limited to a special or customized meaning. The term specifically may, without limitation, refer to the fact that the magnetic force exerted on the movable component at least partially originates from a magnetic attraction and/or a magnetic repulsion between the magnetic driver element and a further magnetic element which is not part of the transdermal medical device, such as the magnetic trigger element of the body mount as described in further detail below. Consequently, the external magnetic force is at least partially constituted by a force between the magnetic driver element of the transdermal medical device and at least one further magnetic element. More specifically, in a rest position or in an unused stage, the transdermal medical device may be configured such that the movable component is in the distal position. The magnetic driver element may then be configured such that the external magnetic force may be an attractive or repellent force driving magnetic driver element and the movable component into the proximal position. As an example and as will be outlined in further detail below, a body mount may be provided on the skin of the user, wherein the body mount comprises a magnetic trigger element, wherein, when the transdermal medical device is attached to the body mount, the magnetic trigger element drives the magnetic driver element and the movable component from the distal position into the proximal position, specifically by magnetically attracting the magnetic driver element.

As outlined above, the stationary component specifically may comprise a sleeve. The sleeve, as an example, may fully or partially be made of a plastic material. The sleeve specifically may be a cylindrical sleeve, such as a cylindrical sleeve having a circular cross-section. The sleeve may be closed on one side, e.g. on a side facing the skin of the user or on the application side. The closure may contain an opening for the penetration element to protrude from the application side, through the opening. On an inner side of the sleeve, at least one guiding surface may be provided along which the movable component may slide. Thus, the movable component may be received within the sleeve. Specifically, the movable component may be slidably received within the sleeve.

As further outlined above, the magnetic driver element may be embodied in various ways, such as by providing a solid permanent magnet or by integrating magnetic particles into a plastic material, e.g. a plastic material of the movable component. Specifically, however, the magnetic driver element may comprise at least one permanent magnet. The permanent magnet specifically may comprise a cylindrical permanent magnet, such as a permanent magnet having the shape of a disk or a ring. The permanent magnet specifically may be one or more of integrated and attached into the movable component. More specifically, the permanent magnet may be attached to the movable component on a side facing the application side. Additionally or alternatively, the permanent magnet may also be received within the movable component, such as within at least one movable inner space within the movable component.

As outlined above, a direction of movement of the movable component from the distal position into the proximal position may be defined as a penetration direction. The transdermal medical device may further comprise at least one retraction spring, the retraction spring being configured for exerting a spring force onto the movable component, the spring force being configured for moving the movable component from the proximal position into the distal position, specifically against the external magnetic force, i.e. into the above-mentioned retraction direction opposing the penetration direction. The external magnetic force may be dimensioned such that the external magnetic force, when the transdermal medical devices is closest to the skin of the user, overcomes the spring force exerted by the retraction spring, whereas, when the transdermal medical device is removed from the body surface, the retraction spring exerts a spring force which overcomes the external magnetic force, such that the movable component is moved back from the proximal position into the distal position. For example, the magnetic trigger element of the body mount as defined in further detail below may be located and dimensioned such that the above-mentioned conditions are fulfilled: when the transdermal medical device is located away from the body mount, the retraction spring may hold the movable component in the distal position, whereas, when the transdermal medical device is attached to the body mount, the external magnetic force exerted by the magnetic trigger element onto the magnetic driver element exceeds the spring force exerted by the retraction spring, so the movable component is moved from the distal position into the proximal position, thereby driving the penetration element into the skin of the user.

The stationary component, as outlined above, may provide at least one receptacle or space, also referred to as a stationary inner space, in which the movable component may move, specifically slide. More specifically, the stationary component may provide at least one sliding surface, such as an inner circumferential surface of a sleeve of the stationary component. The stationary component may comprise one or more further elements which guide and/or limit the movement of the movable component. Specifically, the stationary component may comprise at least one stop defining at least one of the distal position and the proximal position. Specifically, the stationary component may comprise at least two stops defining the distal position and the proximal position. As an example, these at least two stops may be defined by one or more abutment surfaces limiting the movement of the movable component.

As outlined above, the transdermal medical device specifically may be configured for sampling of a bodily fluid, specifically capillary blood. For this purpose, the penetration element may simply create an opening in the skin and/or in the body tissue of the user, specifically also an opening in at least one capillary vessel. From at least one opening, capillary blood or other bodily fluids may simply leak, to be collected manually by the user or by separate sampling elements, such as one or more sampling papers or sampling tubes. However, the transdermal medical device may also comprise at least one integrated element for sampling, such as at least one sampling element configured for receiving at least one sample of the bodily fluid from an incision in the skin of the user created by the penetration element. As an example, the sampling device may comprise at least one sampling tube, specifically a sampling tube received within the movable component. The bodily fluid may be sucked into the sampling device, specifically the sampling tube, by capillary forces or by other forces, such as an underpressure exerted onto the bodily fluid, sucking the bodily fluid into the sampling device. Specifically, the transdermal medical device may further comprise at least one suction element for aspiring at least one sample of the bodily fluid, specifically at least one suction element exerting an underpressure onto the sampling element. As an example, the suction element may comprise a reservoir in which a lower pressure than the pressure of the ambient air may be present, the reservoir being in fluid connection with the sampling element, such that the low pressure in the reservoir sucks the bodily fluid into the sampling element. Additionally or alternatively, the transdermal medical device may also comprise a suction element having an actuator for generating a low pressure or underpressure for sucking the bodily fluid into the sampling device, e.g. an actuator to be operated by the user and/or an automatic actuator which may be coupled to one or more other actions, e.g. the attachment of the transdermal medical device to the body mount, the removal of the transdermal medical device from the body mount or a magnetic action. Other options are feasible.

In addition or as an alternative to the sampling function, with the transdermal medical device being at least partially configured as a sampling device as discussed above, the transdermal medical device may also fully or partially be embodied as an injection device. Thus, the transdermal medical device may be configured for injecting at least one medically active component into the body tissue of the user, specifically through the penetration element. Thus, as an example, the penetration element may be fully or partially configured as an injection needle or as an injection cannula. The transdermal medical device may further comprise at least one reservoir for holding the medically active component. As an example, the reservoir may be or may contain a liquid reservoir for holding one or more liquid medically active components.

As discussed above and as will be discussed in further detail below, the transdermal medical device may be configured for attachment to a body mount. The body mount, specifically, may contain the magnetic trigger element for exerting the external magnetic force onto the transdermal medical device. For the purpose of attachment, the transdermal medical device, on the application side, specifically may comprise at least one device engagement element for engaging with a body mount engagement element of a body mount attached to the body surface of the user. The device engagement element specifically may be configured for mechanically engagement with a mechanical body mount engagement element. More specifically, the engagement may be one or more of a form fit and a force fit connection. The engagement may be suited or configured to hold the transdermal medical device in position on the skin of the user, in one or more than one dimension, such as in a plane locally defined by the body mount or the body surface, such as a tangential plane, whereas, optionally, a movement in a direction perpendicular to that plane may be possible, in order to remove the transdermal medical device from the body mount. As an example, the device engagement element may comprise at least one rim of the stationary component, and the body mount engagement element is receivable within the rim. As an example and as discussed above, the stationary component may comprise a sleeve, with a circular or polygonal rim surrounding an opening into which a corresponding cylindrical body mount engagement element may be inserted. The cylindrical body mount engagement element, as an example and as will be discussed in further detail below, specifically may comprise the magnetic trigger element, such that the rim of the stationary component specifically may, in an engaged state with the transdermal medical device attached to the body mount, fully or partially surround the magnetic trigger element.

In a further aspect of the present invention and as already mentioned above, a body mount as proposed, the body mount being configured for attaching the transdermal medical device according to the present invention to a body surface of a user, e.g. the transdermal medical device according to any one of the embodiments described above and/or according to any one of the embodiments described in further detail below. The body mount comprises at least one application surface for application to the body surface. The body mount further comprises, as already mentioned above, on a side opposing the application surface, at least one body mount engagement element for engaging with the transdermal medical device, specifically with at least one device engagement element of the transdermal medical device. Further, as also already mentioned above, the body mount further comprises at least one magnetic trigger element configured for exerting an external magnetic force to the transdermal medical device in order to magnetically attract the magnetic driver element of the transdermal medical device when the transdermal medical device is attached to the body mount.

For most of the definitions and options of the body mount, reference may be made to the discussion of the transdermal medical device above. The term “body mount”, as used herein, is a broad term and is to be given its ordinary and customary meaning to a person of ordinary skill in the art and is not to be limited to a special or customized meaning. The term specifically may refer, without limitation, to an arbitrary device configured for mounting at least one further device or component to a body surface of a user. The body mount, as an example, may comprise one or more fixation elements for fixating the body mount to the body surface, e.g. one or more adhesives. Thus, as an example, the body mount may fully or partially be embodied as an adhesive plaster. In order to mount the one or more further devices or components to the body surface of the user, the body mount comprises the body mount engagement element.

In the specific embodiments described herein, the body mount is configured for attaching the transdermal medical device to the body surface. Thus, the body mount specifically may act as an intermediate component in between the transdermal medical device and the body surface of the user, with the body mount being attached, with the application surface, to the body surface, and with the body mount further, on a side facing away from the body surface, being connectable or attachable to the transdermal medical device.

The term “application surface”, as used herein, is a broad term and is to be given its ordinary and customary meaning to a person of ordinary skill in the art and is not to be limited to a special or customized meaning. The term specifically may refer, without limitation, to a surface of an element with which we element may be applied to a target element. In the present case, the application surface specifically is a surface which directly or indirectly, e.g. via one or more adhesive layers, is in contact with the body surface of the user. The application surface specifically may contain one or more adhesives.

The terms “device engagement element” and “body mount engagement element”, respectively, as used herein, are broad terms and are to be given their ordinary and customary meaning to a person of ordinary skill in the art and is not to be limited to a special or customized meaning. The terms specifically may refer, without limitation, to elements of the transdermal medical device or the body mount, respectively, which are configured for engaging with each other, in order to attach the transdermal medical device to the body mount, specifically reversibly. Thus, the engagement elements generally may comprise corresponding mechanical structures which are configured for mechanically interacting with each other, in order to provide a connection between the transdermal medical device and the body mount, which specifically may be suited to hold the transdermal medical device in a dedicated position on the body mount, in one or more than one dimension, such as, as explained above, in a plane defined by the body surface or by the body mount. More specifically, the connection may be a reversible mechanical connection. Thus, as an example, the engagement elements may comprise complementary mechanical structures which are configured for engaging with each other, e.g. according to the key-lock principle. As discussed above, as an example, the device engagement element may comprise a circumferential rim of the stationary component of the transdermal medical device, whereas the body mount engagement element may comprise a corresponding protrusion on the body mount which fits into the opening defined by the rim, specifically with a low mechanical tolerance in order to provide a precise positioning of the transdermal medical device on the body mount. As further discussed above, the protrusion on the body mount specifically may be formed by or may at least contain the magnetic trigger element.

As also discussed above, the body mount further comprises at least one magnetic trigger element, the magnetic trigger element being configured for exerting an external magnetic force to the transdermal medical device in order to magnetically attract the magnetic driver element of the transdermal medical device when the transdermal medical device is attached to the body mount. The term “magnetic trigger element”, as used herein, is a broad term and is to be given its ordinary and customary meaning to a person of ordinary skill in the art and is not to be limited to a special or customized meaning. The term specifically may refer, without limitation, to an arbitrary magnetic element which is configured for exerting an external magnetic force on to the transdermal medical device, specifically onto the magnetic driver element of the movable component, specifically in order to move the movable component from the distal position into the proximal position.

The magnetic trigger element specifically may comprise at least one magnetic material, such as at least one permanent magnet. The magnetic material may be formed or may form a permanent magnet or, alternatively, may also be integrated into one or more other components, such as by the magnetic driver element comprising magnetic particles comprised as a filler material in a plastic material. The magnetic trigger element specifically may also be embodied as a ring-shaped magnet. The ring-shaped magnet may be configured for resting on the body surface of the user, wherein the body mount engagement element may fully or partially be formed by the ring-shaped magnet and wherein the penetration element of the transdermal medical device attached to the body mount may protrude, in the proximal position, through the central opening of the ring-shaped magnet into the body tissue of the user. Thus, specifically, the application surface of the body mount may also fully or partially be formed by the magnetic trigger element, e.g. by a ring-shaped front face of the magnetic trigger element resting on the skin of the user. Various alternatives are feasible.

The magnetic trigger element may be integrated or attached to the body mount, such as into a base or frame of the body mount, e.g. a base or frame forming the application surface. Thus, as an example, the body mount may contain at least one base element, the base element e.g. forming or having the application surface, wherein the magnetic trigger element is one or both of mounted to and integrated into the base element, wherein, specifically, the magnetic trigger element may form a protrusion protruding from the base element on a side facing away from the body surface. The base element, however, may also at least partially and specifically fully be formed by the magnetic trigger element.

As also outlined above, the body mount specifically may be configured for attachment to the body surface of the user. More specifically, the body mount may further comprise at least one adhesive element for attaching the application surface to the body surface of the user, specifically at least one adhesive plaster. As outlined above, the magnetic trigger element specifically may comprise at least one permanent magnet, such as a cylindrical ring magnet or ring-shaped magnet, specifically a permanent magnet attached to the adhesive plaster. The penetration element, during penetration, may move through a central opening of the ring-shaped magnet and optionally through an opening in the plaster, through the skin of the user and into the body tissue.

As further outlined above, the body mount engagement element may fully or partially be integrated with the magnetic trigger element, specifically with the permanent magnet. Thus, the permanent magnet itself may protrude from the body surface may engage with e.g. the stationary component and the device engagement element, such as an opening surrounded by the rim of the stationary component. Thus, specifically, the magnetic trigger element may be a ring-shaped magnetic trigger element, and an opening defined by the rim of the stationary component may be dimensioned such that the ring-shaped magnetic trigger element fits within the opening.

In a further aspect of the present invention, a transdermal medical kit is proposed. The transdermal medical kit comprises at least one transdermal medical device according to the present invention, such as according to any of the embodiments described above or any of the embodiments described in further detail below. Further, the transdermal medical kit comprises at least one body mount according to the present invention, such as according to any one of the embodiments described above and/or according to any one of the embodiments described in further detail below.

The term “kit”, as used herein, is a broad term and is to be given its ordinary and customary meaning to a person of ordinary skill in the art and is not to be limited to a special or customized meaning. The term specifically may refer, without limitation, to a plurality of components being configured to interact to fulfill at least one purpose. Consequently, the term “medical kit”, as used herein, is a broad term and is to be given its ordinary and customary meaning to a person of ordinary skill in the art and is not to be limited to a special or customized meaning. The term specifically may refer, without limitation, to a kit configured to fulfill at least one medical purpose, such as a diagnostic purpose and/or a therapeutic purpose. Further and further consequently, the term “transdermal medical kit”, as used herein, also is a broad term and is to be given its ordinary and customary meaning to a person of ordinary skill in the art and is not to be limited to a special or customized meaning. The term specifically may refer, without limitation, to a medical kit which is configured for performing at least one transdermal action, as defined above.

In the transdermal medical kit, the transdermal medical device specifically may comprise at least one device engagement element as defined above. The device engagement element and the body mount engagement element may be configured to interact such that the transdermal medical device is aligned, specifically self-aligned, more specifically self-centered, when the transdermal medical device is attached to the body mount. This alignment, specifically the self-alignment or even self-positioning, may specifically be performed by the above-mentioned option in which the magnetic trigger element is formed as a cylindrical magnet, specifically as a cylindrical ring magnet, which may directly or indirectly be mounted to the body surface, and wherein the magnetic trigger element forms or forms part of the body mount engagement element. Further, as also discussed above, the device engagement element may fully or partially be formed by a rim of the transdermal medical device, specifically a rim of the stationary component of the transdermal medical device, wherein an opening surrounded by the rim may be dimensioned such that the magnetic trigger element may be inserted into the opening.

As also outlined above, the magnetic trigger element and the magnetic driver element specifically may be configured such that a penetration movement of the movable component is triggered by the magnetic trigger element when the body mount engagement element fully engages with the transdermal medical device, specifically with the device engagement element of the transdermal medical device.

The transdermal medical kit may, as outlined above, comprise one or more further elements. Thus, as an example, the transdermal medical kit and/or the transdermal medical device may comprise one or more casings or packagings which may fully or partially enclose the transdermal medical device and the body mount. Further, the transdermal medical kit and/or the transdermal medical device may comprise one or more protective devices, e.g. for holding the movable component in a safe position, e.g. in the distal position, before use, such as during storage for shipment. The protective device, as an example, may be configured for blocking a movement of the movable component from the distal position into the proximal position. The user may remove the protective device before use. The protective device, as an example, may comprise a clamp, a pin or the like, in order to secure the transdermal medical device.

In a further aspect of the present invention, a method of driving at least one penetration element is proposed. The method comprises the following steps which specifically may be performed in the given order. A different order, however, is also feasible. Further, two or more or even all of the method steps may be performed sequentially or overlapping in time or even simultaneously. Further, one or more or even all of the method steps may be performed once or repeatedly. The method may contain additional method steps which are not listed.

The method comprises the following steps:
a. providing a transdermal medical device, the transdermal medical device comprising at least one stationary component and at least one movable component, wherein the movable component is mounted to the stationary component in a movable manner relative to the stationary component, wherein the movable component is movable from a distal position to a proximal position, wherein the stationary component comprises an application side, wherein the movable component comprises the at least one penetration element configured for penetrating the skin of a user, wherein the penetration element, in the distal position of the movable component, is received within the stationary component and wherein the penetration element, in the proximal position of the movable component, protrudes from the application side of the stationary component, wherein the movable component further comprises at least one magnetic driver element, wherein the magnetic driver element is configured such that a movement of the movable component is drivable by at least one external magnetic force;
b. providing a body mount configured for attaching the transdermal medical device to a body surface of a user, wherein the body mount comprises at least one application surface for application to the body surface, wherein the body mount further comprises, on a side opposing the application surface, at least one body mount engagement element for engaging with the transdermal medical device, specifically with at least one device engagement element of the transdermal medical device, and wherein the body mount further comprises at least one magnetic trigger element;
c. attaching the body mount to a skin of the user; and
d. attaching the transdermal medical device to the body mount, wherein the transdermal medical device engages with the body mount engagement element, wherein the magnetic trigger element of the body mount exerts an external magnetic force to the transdermal medical device, thereby magnetically attracting the magnetic driver element of the transdermal medical device.

Specifically, the method may make use of one or more of the transdermal medical device, the body mount and the transdermal medical kit according to the present invention, such as according to any one of the embodiments described above and/or according to any one of the embodiments described in further detail below. Thus, for possible embodiments of the method, as well as for possible definitions, reference may be made to the description of the transdermal medical device, the body mount or the transdermal medical kit.

The transdermal medical device, the body mount, the transdermal medical kit and to the method of driving the penetration element, as proposed herein, provide a large number of advantages over known devices, systems and methods of similar kind.

Thus, specifically, the above-mentioned technical challenges of known means and methods are efficiently addressed. The devices and the method according to the present invention specifically may provide for a reproducible and precise depth of puncturing our penetration into the body tissue of the user. Thereby, inconvenience for the user may efficiently be reduced. Still, by providing the reliable depth of puncturing, a sufficient amount of blood having a high quality may be produced. Further, in view of the simple handling and, in view of the reproducible amount of blood generated, contamination of the user's personal belongings may be avoided.

Further, the invention efficiently addresses psychological issues involved in home care, self-testing, or self-monitoring. Thus, since the process of puncturing basically may take place automatically when the user attaches the transdermal medical device to the body mount, the penetration of the skin requires a lower self-overcoming of the user. The process may be kept simple and convenient, so samples of bodily fluid may be generated and prepared for further processing and analysis in a fast, reliable, clean, convenient and efficient manner. The sample may then be processed according to the specific requirements of the situation, e.g. through analysis by the user herself or himself, or by shipping the sample to an appropriate analysis facility.

From a technical point of view, the invention also provides a large number of advantages over known devices and methods of similar kind. Specifically, the transdermal device may be designed such that no pressure onto the skin or body surface of the user is required for triggering. The triggering simply takes place, by attaching the transdermal device with the body mount, which may take place without any pressure. Thereby, a compression or squeezing of the body tissue may be avoided, which, as outlined above, may have detrimental effects on the reproducibility of the puncturing depth. This is basically due to the fact that the compression of the body tissue typically is dependent on the constitution of the body tissue, e.g. the softness, the content of water, the body fat content and the like. Consequently, in conventional systems, exerting pressure onto the body tissue, the compression of the body tissue, and, thereby, the puncturing depth may vary even though the pressure onto the body tissue may be identical. With the present invention using a magnetic triggering mechanism which may avoid the exertion of pressure, this problem may at least partially be overcome.

Further, with the present invention, a positioning of the puncturing site may be simplified. The body mount may be placed precisely by the user, over the desired site of puncturing, e.g. by using the ring-shaped magnet of the body mount as a positioning aid. Additionally or alternatively, other positioning aids may also be used and may also be present in the body mount, such as fiducial marks or the like. Since the applying of the transdermal medical device to the body mount is supported by magnetic attraction, the invention may imply a self-centering or self-positioning of the transdermal medical device on the body mount.

Summarizing and without excluding further possible embodiments, the following embodiments may be envisaged:
Embodiment 1: A transdermal medical device, comprising at least one stationary component and at least one movable component, wherein the movable component is mounted to the stationary component in a movable manner relative to the stationary component, wherein the movable component is movable from a distal position to a proximal position, wherein the stationary component comprises an application side, wherein the movable component comprises at least one penetration element configured for penetrating the skin of a user, wherein the penetration element, in the distal position of the movable component, is received within the stationary component and wherein the penetration element, in the proximal position of the movable component, protrudes from the application side of the stationary component, wherein the movable component further comprises at least one magnetic driver element, wherein the magnetic driver element is configured such that a movement of the movable component is drivable by at least one external magnetic force.
Embodiment 2: The transdermal medical device according to the preceding embodiment, wherein the stationary component comprises a sleeve, specifically a cylindrical sleeve.
Embodiment 3: The transdermal medical device according to the preceding embodiment, wherein the movable component is slidably received within the sleeve.
Embodiment 4: The transdermal medical device according to any one of the preceding embodiments, wherein the magnetic driver element comprises a permanent magnet.
Embodiment 5: The transdermal medical device according to the preceding embodiment, wherein the permanent magnet comprises a cylindrical permanent magnet.
Embodiment 6: The transdermal medical device according to any one of the two preceding embodiments, wherein the permanent magnet is attached to the movable component on a side facing the application side.
Embodiment 7: The transdermal medical device according to any one of the preceding embodiments, further comprising at least one retraction spring, the retraction spring being configured for exerting a spring force onto the movable component, the spring force being configured for moving the movable component from the proximal position into the distal position, specifically against the external magnetic force.
Embodiment 8: The transdermal medical device according to the preceding embodiment, wherein the spring force of the retraction spring is configured to overcome the external magnetic force, such that the movable component is moved back from the proximal position into the distal position, when the transdermal medical device is removed from the body surface, specifically when the transdermal medical device is detached from the body mount.
Embodiment 9: The transdermal medical device according to any one of the preceding embodiments, wherein the stationary component comprises at least one stop defining at least one of the distal position and the proximal position, specifically one stop defining the distal position and one stop defining the proximal position.
Embodiment 10: The transdermal medical device according to any one of the preceding embodiments, wherein the transdermal medical device is configured for sampling of a bodily fluid, specifically capillary blood, wherein the transdermal medical device further comprises at least one sampling element, wherein the sampling element is configured for receiving at least one sample of the bodily fluid from an incision in the skin of the user created by the penetration element.
Embodiment 11: The transdermal medical device according to the preceding embodiment, wherein the sampling device comprises at least one sampling tube, specifically a sampling tube received within the movable component.
Embodiment 12: The transdermal medical device according to any one of the two preceding embodiments, wherein the transdermal medical device further comprises at least one suction element for aspiring at least one sample of the bodily fluid, specifically at least one suction element exerting an underpressure onto the sampling element.
Embodiment 13: The transdermal medical device according to any one of the preceding embodiments, wherein the transdermal medical device is configured for injecting at least one medically active component into the body tissue of the user, specifically through the penetration element.
Embodiment 14: The transdermal medical device according to the preceding embodiment, wherein the transdermal medical device further comprises at least one reservoir for holding the medically active component.
Embodiment 15: The transdermal medical device according to any one of the preceding embodiments, wherein the transdermal medical device, on the application side, comprises at least one device engagement element for engaging with a body mount engagement element of a body mount attached to the body surface of the user.
Embodiment 16: The transdermal medical device according to the preceding embodiment, wherein the device engagement element comprises at least one rim of the stationary component, wherein the body mount engagement element is receivable within the rim.
Embodiment 17: A body mount for attaching the transdermal medical device according to any one of the preceding embodiments to a body surface of a user, wherein the body mount comprises at least one application surface for application to the body surface, wherein the body mount further comprises, on a side opposing the application surface, at least one body mount engagement element for engaging with the transdermal medical device, specifically with at least one device engagement element of the transdermal medical device, and wherein the body mount further comprises at least one magnetic trigger element, the magnetic trigger element being configured for exerting an external magnetic force to the transdermal medical device in order to magnetically attract the magnetic driver element of the transdermal medical device when the transdermal medical device is attached to the body mount.
Embodiment 18: The body mount according to the preceding embodiment, further comprising at least one adhesive element for attaching the application surface to the body surface of the user, specifically at least one adhesive plaster.
Embodiment 19: The body mount according to any one of the preceding embodiments referring to a body mount, wherein the magnetic trigger element comprises at least one permanent magnet, specifically a permanent magnet attached to the adhesive plaster.
Embodiment 20: The body mount according to the preceding embodiment, wherein the permanent magnet comprises a cylindrical ring magnet.
Embodiment 21: The body mount according to any one of the preceding embodiments referring to a body mount, wherein the body mount engagement element is at least partially integrated with the magnetic trigger element, specifically with the permanent magnet.
Embodiment 22: A transdermal medical kit, comprising at least one transdermal medical device according to any one of the preceding embodiments referring to a transdermal medical device and, further comprising at least one body mount according to any one of the preceding embodiments referring to a body mount.
Embodiment 23: The transdermal medical kit according to the preceding embodiment, wherein the device engagement element and the body mount engagement element are configured to interact such that the transdermal medical device is aligned, specifically self-aligned, more specifically self-centered, when the transdermal medical device is attached to the body mount.
Embodiment 24: The transdermal medical kit according to any one of the preceding embodiments referring to a transdermal medical kit, wherein the magnetic trigger element and the magnetic driver element are configured such that a penetration movement of the movable component is triggered by the magnetic trigger element when the body mount engagement element fully engages with the transdermal medical device, specifically with the device engagement element of the transdermal medical device.
Embodiment 25: A method of driving at least one penetration element, the method comprising:
   a. providing a transdermal medical device, the transdermal medical device comprising at least one stationary component and at least one movable component, wherein the movable component is mounted to the stationary component in a movable manner relative to the stationary component, wherein the movable component is movable from a distal position to a proximal position, wherein the stationary component comprises an application side, wherein the movable component comprises the at least one penetration element configured for penetrating the skin of a user, wherein the penetration element, in the distal position of the movable component, is received within the stationary component and wherein the penetration element, in the proximal position of the movable component, protrudes from the application side of the stationary component, wherein the movable component further comprises at least one magnetic driver element, wherein the magnetic driver element is configured such that a movement of the movable component is drivable by at least one external magnetic force;
   b. providing a body mount configured for attaching the transdermal medical device to a body surface of a user, wherein the body mount comprises at least one application surface for application to the body surface, wherein the body mount further comprises, on a side opposing the application surface, at least one body mount engagement element for engaging with the transdermal medical device, specifically with at least one device engagement element of the transdermal medical device, and wherein the body mount further comprises at least one magnetic trigger element;
   c. attaching the body mount to a skin of the user; and
   d. attaching the transdermal medical device to the body mount, wherein the transdermal medical device engages with the body mount engagement element, wherein the magnetic trigger element of the body mount exerts an external magnetic force to the transdermal medical device, thereby magnetically attracting the magnetic driver element of the transdermal medical device.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an explosion drawing of an embodiment of a transdermal medical kit;

FIG. 2 shows a cross-sectional view of the transdermal medical device of the transdermal medical kit of FIG. 1 in an initial state;

FIG. 3 shows a cross-sectional view of the transdermal medical device of FIG. 2, in a triggered state, after attachment to a body mount of the transdermal medical kit of FIG. 1;

FIG. 4 shows a perspective view of the transdermal medical device of FIGS. 1 to 3, with an additional protective device; and

FIG. 5 shows a flowchart of an embodiment of a method of driving at least one penetration element.

### DETAILED DESCRIPTION OF THE EMBODIMENTS

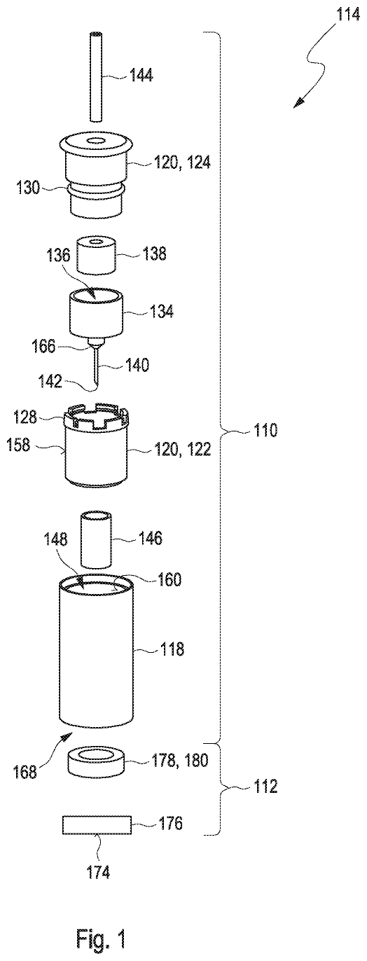

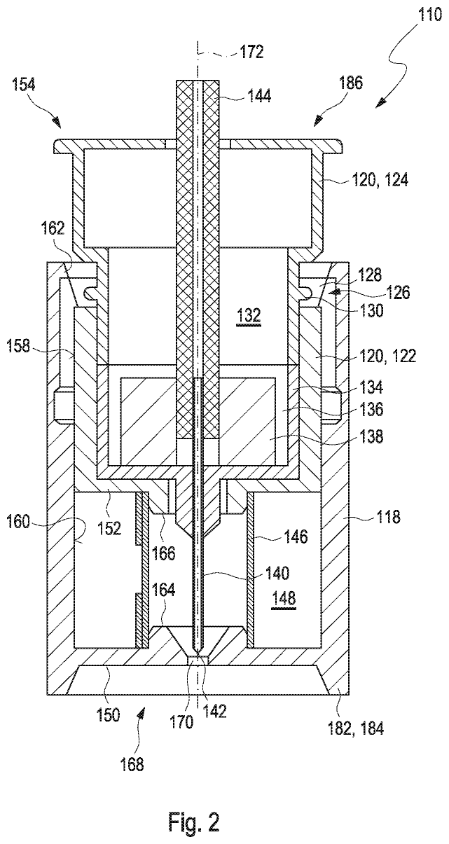

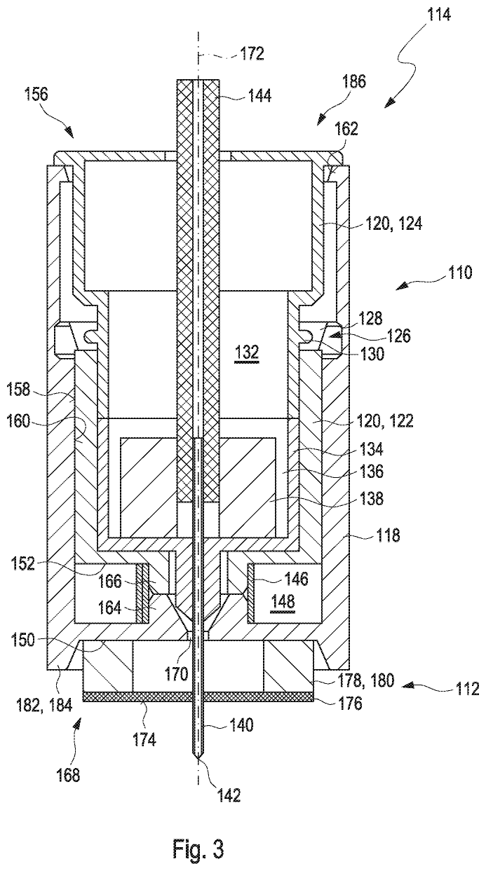

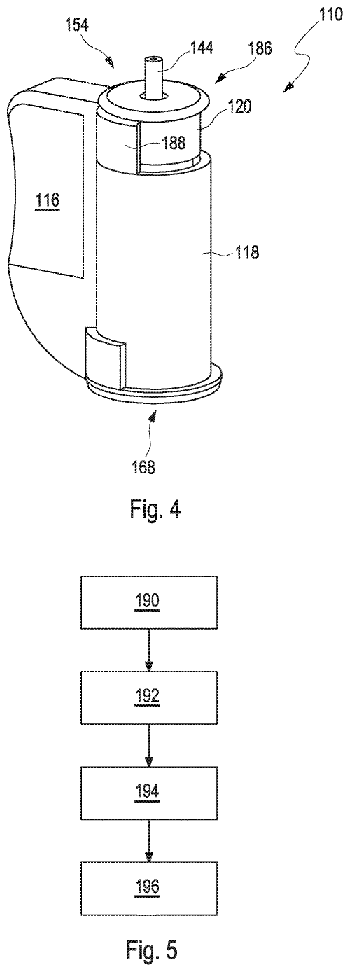

In FIGS. 1 to 4, an exemplary embodiment of a transdermal medical device 110, of a body mount 112 and of a transdermal medical kit 114 is shown in various views. Therein, FIG. 1 is an explosion drawing of the transdermal medical kit 114. FIG. 2 shows the transdermal medical device 110 in an initial state, in a cross-sectional view. FIG. 3 shows the transdermal medical device 110 after attachment to the body mount 112, in a triggered state, also as a cross-sectional view. FIG. 4 shows the transdermal medical device 110 in a perspective view, with an additional protective device 116, which acts as a safety device. These figures, in the following, will be described in conjunction.

The transdermal medical kit 114, as shown in the explosion drawing of FIG. 1, comprises the transdermal medical device and the body mount 112 to be attached to a body surface of a user. The transdermal medical device 110, comprises a stationary component 118 and a movable component 120. The movable component 120, in this embodiment, specifically may comprise two elements, which is a movable lower part 122 and a movable upper part 124. The movable lower part 122 and the movable upper part 124 are formed as hollow parts, specifically as hollow sleeves, and are nested into each other and joined by a connection, such as a snap fit connection 126, as can be seen, e.g. in FIG. 2 or 3. The snap fit connection 126, as an example, may be formed by a crimped rim 128 of the movable lower part 122 and by a circumferential bead 130 of the movable upper part 124.

The movable lower part 122 and the movable upper part 124 surround a movable inner space 132, as can be seen in FIGS. 2 and 3. In this movable inner space 132, in this embodiment, a needle holder 134 may be embodied, e.g. clamped down and held in place, by the movable upper part 124. The needle holder 134, as an example, may surround a needle holder space 136 and, as an example, may be formed as a shell and/or may have the shape of a cup, as shown in FIGS. 1 to 3. In this embodiment, within the needle holder space 136, a magnetic driver element 138 is received and mounted, which, as an example, may have the form of a ring-shaped permanent magnet.

The needle holder 134, on its bottom side, holds a penetration element 140. The penetration element 140, as an example, may have the shape of a hollow needle or a cannula. The penetration element 140, as an example, may have a pointy end, a tip 142 or a sharp, on its proximal end.

The transdermal medical device 110 specifically may further comprise at least one sampling element 144. In the embodiment shown in FIGS. 1 to 4, the sampling element 144 specifically may have the shape of a tube. The sampling element 144 may be in fluid connection with a channel within the penetration element 140, such as a lumen of the cannula or hollow needle. The sampling element 144 specifically may be received within the movable component 120.

The transdermal medical device 110 further may comprise at least one retraction spring 146. The retraction spring 146, as an example, may be located in a stationary inner space 148 formed by the stationary component 118. Thus, as an example, the stationary component 118 may have the shape of a cylindrical sleeve or shell, with the movable component 120 received therein. The retraction spring 146 may be interposed, in the stationary inner space 148, in between a front plate 150 of the stationary component 118 and a front plate 152 of the movable component 120.

The movable component 120 is mounted to the stationary component 118, e.g. by being received within the stationary inner space 148, in a movable manner relative to the stationary component 118. Thus, as can be seen by comparing FIGS. 2 and 3, the movable component 120 is movable from a distal position 154, as shown in the configuration of FIG. 2, to a proximal position 156, as depicted in FIG. 3. The movement specifically may take place in a sliding fashion, with a circumferential outer surface 158 of the movable component 120 gliding over a circumferential inner surface 160 of the sleeve of the stationary component 118. The movement of the movable component 120 may be limited by at least one stop. In the exemplary embodiment shown, the stationary component 118 may provide for an upper stop 162 and further, optionally, for a lower stop 164. As an example, the upper stop 162 may be formed by a crimped rim of the sleeve of the stationary component 118, with a part of the movable component 120, e.g. an upper side of the crimped rim 128, abutting the rim, thereby defining the distal position of the movable component 120. The lower stop 164, as an example, may be formed by a protruding part of the front plate 150 of the stationary component 118, protruding into the stationary inner space 148. The proximal position 156, as an example, may be defined by a protruding lower rim 166 of the movable component 120 abutting the protruding lower rim 166. The protruding rim 166, as an example, may be an injection molded protrusion which is used for holding the penetration element 142 the movable component 120. It shall be noted, however, that many other technical realizations of the upper and lower stops 162, 164 are feasible. Thus, as an example, the lower stop 164 may also be realized simply by the retraction spring 146 being fully compressed.

The stationary component 118 has an application side 168. With this application side 168, the transdermal medical device 110 may face the skin of the user, during penetration of the user's skin. As shown in FIGS. 2 and 3, the stationary component 118 may have, specifically within the front plate 150, an opening 170. Specifically, the opening 170, along with the penetration element 140, may be located on a penetration axis 172, which may also form an axis of symmetry of the transdermal medical device 110 and which may also define a direction of movement of the movable component 120. As outlined above, the movable component 120 may move from the distal position 154 to the proximal position 156 and, optionally, back into the distal position 154. When in the distal position 154, the penetration element 140 specifically may be fully received within the stationary inner space 148 of the stationary component 118. When in the proximal position 156, the penetration element 140 protrudes from the application side 168 of the transdermal medical device 110. Thus, specifically, in the embodiment shown in FIGS. 2 and 3, the penetration element 140 may protrude through the opening 170, on the application side 168.

The transdermal medical kit 114, as outlined above, besides the transdermal medical device 110, further comprises the body mount 112. The body mount 112 is configured for attaching the transdermal medical device 110 to the body surface of the user. For this purpose, the body mount 112 may comprise an application surface 174. For attaching the body mount 112 to the body surface, the application surface 174 specifically may comprise at least one adhesive 176. Thus, as an example, the body mount 112, specifically the application surface 174 of the body mount 112, may comprise an adhesive plaster, optionally with a detachable liner, which may be attached to the body surface by adhesive forces, before assembling the entire transdermal medical kit 114 by attaching the transdermal medical device 110 to the body mount 112.

The body mount 112 further comprises at least one magnetic trigger element 178. As an example, the magnetic trigger element may comprise a ring-shaped permanent magnet. When attaching the transdermal medical device 110 to the body mount 112, the magnetic trigger element 178 is configured and oriented such that an external magnetic force is exerted onto the transdermal medical device 110, specifically to the magnetic driver element 138. Specifically, the external magnetic force is oriented such that the magnetic trigger element 178 attracts the magnetic driver element 138 of the transdermal medical device 110, thereby moving the movable component 120, from the distal position 154 shown in FIG. 2, against the force of the retraction spring 146, into the proximal position 156, as shown in FIG. 3. Thus, generally, the magnetic fields generated by the magnetic driver element 138 and the magnetic trigger element 178 may be oriented in the same direction, at least as far as their components parallel to the penetration axis 172 are concerned.

The transdermal medical device 110 is configured for being attached to the body mount 112. For this purpose, the body mount 112 may comprise at least one body mount engagement element 180, specifically a body mount engagement element 180 configured for mechanically engaging with a device engagement element 182 of the transdermal medical device 110. In the embodiment shown in FIGS. 1 and 3, in which the body mount 112 is shown, the body mount simply may have an upper circumferential rim, e.g. a circular rim, which may engage with a corresponding rim 184 of the stationary component 118. Thus, rim 184 may protrude over the front plate 150, thereby forming a receptacle dimensioned to receive the outer rim of the ring-shaped magnet of the magnetic trigger element 178. Engagement elements 180, 182 may be configured to enable self-positioning or self-centering. Thus, as shown in FIGS. 2 and 3, the receptacle of the stationary component 118 may have a conical shape, by providing inclined circumferential inner walls.

As visible in FIGS. 2 and 3, the movable component 120, in the unused state of the transdermal medical device 110, with the movable component 120 being in the distal position 154, may protrude from the stationary component 118 on a distal side 186 opposing the application side 168. The protective device 116 may comprise a clamp 188 engaging with the movable component 120, such that the movable component 120 is held in place, in the distal position 154, without being able to move into the proximal position. Thereby, an unwanted triggering of the transdermal medical device 110 may be prevented, e.g. during shipment or storage.

For assembly of the transdermal medical device 110, in the embodiment of FIGS. 1 to 3, firstly, the retraction spring 146 may be placed into the stationary inner space 148 of the stationary component 118. Subsequently, the movable component 120 is placed into the stationary inner 148. The movable component 120 itself may be assembled by receiving the magnetic driver element 138 in the movable component 120, e.g. by inserting the magnetic driver element 138, e.g. the ring-shaped magnet, in the needle holder space 136 of the needle holder 134. The needle holder 134, with the magnetic driver element 138 inserted therein, may be inserted into the movable in a space 132, specifically by inserting the needle holder 134, with the magnetic driver element 138 inserted therein, into the cup-shaped movable lower part 122 of the movable component 120. Subsequently, the movable upper part 124 of the movable component 120 may be joint with the movable lower part 122 of the movable component 120, e.g. by the snap fit connection 126.

The application side 168 of the transdermal medical device 110, before use, may be closed and/or sealed. Thus, as an example, the application side 168 may fully or partially be covered with a detachable liner or detachable seal which is not depicted in the Figures and which may be removed before use.

In use, the body mount 112 of the transdermal medical kit 114 is placed on the desired part of the body surface of the user, e.g. by using adhesive 176. For this purpose, as an example, a detachable liner may be detached from the body mount 112, specifically from the adhesive 176 on the application surface 174.

Subsequently, the transdermal medical device 110 may be moved to the body mount 112. Specifically, during this approaching, a self-centering of the transdermal medical device 110 with respect to the body mount 112 may take place, such that, as an example, rotational axes of the transdermal medical device 110, e.g. penetration axis 172, and of the body mount 112, e.g. an axis of rotation of the ring-shaped magnet, align, specifically automatically.

Further, during approaching and attaching the transdermal medical device 110 to the body mount 112, the transdermal medical device 110 is triggered by magnetic forces. This is due to the fact that the magnetic trigger element 178 of the body mount 112 exerts an attractive external magnetic force on to the magnetic driver element 138 of the transdermal medical device 110. Thereby, the movable component 120 of the transdermal medical device is pulled, from the distal position 154, into the proximal position 156, against the force of the retraction spring 146 which is compressed during this triggering process. Thereby, penetration element 140 emerges from the opening 170, penetrates the skin of the user and enters the body tissue of the user by a well-defined puncturing depth, mostly defined by the geometric protrusion of the penetration element 140 over application surface 174 in FIG. 3. Ideally, this process does not require any exertion of pressure onto the skin of the user, so a compression of the skin and the body tissue is avoided.

After penetration, bodily fluid, e.g. capillary blood, may enter a hollow lumen of the penetration element 140, e.g. the hollow needle or the capillary. From there, the sample of the bodily fluid may enter the sampling element 144, e.g. the lumen of the tube.

After successful sampling, the transdermal medical device 110 may be detached from the body mount 112. This detachment simply may take place by removing the medical device 110 in an axial direction, from the body surface. During that removal, the external magnetic force exerted by the magnetic trigger element 178 onto the magnetic driver element 138 is continually lowered, and the spring force of the retraction spring 146 pushes the movable component 120 back into the distal position 154, whereby the penetration element 140 is further removed from the body tissue, back into the stationary inner space 148 of the transdermal medical device 110.

The transdermal medical kit 114 as shown in the exemplary embodiments provide for an automatic triggering during attachment of the transdermal medical device 110 to the body mount 112. Further, as discussed above, a precise positioning of the incision side is possible, mainly due to the self-positioning or self-centering. Further, a precise control of the penetration depth or puncturing depth is feasible. The size and the weight of the transdermal medical kit 114 may be adapted to the actual needs.

The transdermal medical kit 114 as shown in FIGS. 1 to 4 may be varied and modified in various ways. Thus, as an example, the inner structure and assembly of the stationary and movable components 118, 120 may vary. Further, the attachment and/or integration of the magnetic driver element 138 may also deviate from the embodiments shown. Further, the sampling element 144 may also be embodied deviating from the embodiments shown, as may the purpose of the transdermal medical kit 114 and the transdermal medical device 110. As an example, in addition or as an alternative to sampling of bodily fluids, the transdermal medical kit 114 may also be used for injection purposes, as discussed above. Finally, the embodiment of the retraction spring 146, as well as the embodiment of the engagement elements 180, 182 may also vary. These are just some examples of possible modifications of the transdermal medical device 110, the body mount 112 at the transdermal medical kit 114, which all are feasible within the scope of the present invention.

In FIG. 5, a generalized flow chart of a method of driving at least one penetration element 140 is proposed. The method, as an example, may be applied to the embodiments shown in FIGS. 1 to 4. Other embodiments, however, are also feasible. The method comprises the steps shown in FIG. 5, which specifically may be performed in the given order. A different order, however, is also feasible. Further, two or more or even all of the method steps may be performed sequentially or overlapping in time or even simultaneously. Further, one or more or even all of the method steps may be performed once or repeatedly. The method may contain additional method steps which are not listed.

The method comprises the following steps:
a. (denoted by reference number 190) providing a transdermal medical device 110, the transdermal medical device 110 comprising at least one stationary component 118 and at least one movable component 120, wherein the movable component 120 is mounted to the stationary component 118 in a movable manner relative to the stationary component 118, wherein the movable component 120 is movable from a distal position 154 to a proximal position 156, wherein the stationary component 118 comprises an application side 168, wherein the movable component 120 comprises the at least one penetration element 140 configured for penetrating the skin of a user, wherein the penetration element 140, in the distal position 154 of the movable component 120, is received within the stationary component 118 and wherein the penetration element 140, in the proximal position 156 of the movable component 120, protrudes from the application side 168 of the stationary component 118, wherein the movable component 120 further comprises at least one magnetic driver element 138, wherein the magnetic driver element 138 is configured such that a movement of the movable component 120 is drivable by at least one external magnetic force;
b. (denoted by reference number 192) providing a body mount 112 configured for attaching the transdermal medical device 110 to a body surface of a user, wherein the body mount 112 comprises at least one application surface 174 for application to the body surface, wherein the body mount 112 further comprises, on a side opposing the application surface, at least one body mount engagement element 180 for engaging with the transdermal medical device 110, specifically with at least one device engagement element 182 of the transdermal medical device 110, and wherein the body mount 112 further comprises at least one magnetic trigger element 178;
c. (denoted by reference number 194) attaching the body mount 112 to the skin of the user; and
d. (denoted by reference number 196) attaching the transdermal medical device 110 to the body mount 112, wherein the transdermal medical device 110 engages with the body mount engagement element 180, wherein the magnetic trigger element 178 of the body mount 112 exerts an external magnetic force to the transdermal medical device 110, specifically to the magnetic driver element 138 of the transdermal medical device 110, thereby magnetically attracting the magnetic driver element 138 of the transdermal medical device 110.

LIST OF REFERENCE NUMBERS
110 Transdermal medical device
112 Body mount
114 Transdermal medical kit
116 Protective device
118 Stationary component
120 Movable component
122 Movable lower part
124 Movable upper part
126 Snap-fit connection
128 Crimped rim
130 Circumferential bead
132 Movable inner space
134 Needle holder
136 Needle holder space
138 Magnetic driver element
140 Penetration element
142 Tip
144 Sampling element
146 Retraction spring
148 Stationary inner space
150 Front plate
152 Front plate
154 Distal position
156 Proximal position
158 Circumferential outer surface
160 Circumferential inner surface
162 Upper stop
164 Lower stop
166 Lower rim
168 Application side
170 Opening
172 Penetration axis
174 Application surface
176 Adhesive
178 Magnetic trigger element
180 Body mount engagement element
182 Device engagement element
184 Rim
186 Distal side
188 Clamp
190 providing a transdermal medical device
192 providing a body mount
194 attaching the body mount to the skin of the user
196 attaching the transdermal medical device to the body mount

What is claimed is:

1. A transdermal medical device, comprising a stationary component and a movable component, wherein:
the movable component is mounted to the stationary component in a movable manner relative to the stationary component,
the movable component is movable from a distal position to a proximal position, wherein the stationary component comprises an application side,
the movable component comprises a penetration element configured for penetrating skin of a user, wherein the penetration element, in the distal position of the movable component, is received within the stationary component and wherein the penetration element, in the proximal position of the movable component, protrudes from the application side of the stationary component, and
the movable component further comprises a magnetic driver element, wherein the magnetic driver element is configured such that a movement of the movable component is drivable by at least one external magnetic force.

2. The transdermal medical device of claim 1, wherein the stationary component comprises a sleeve, and wherein the movable component is slidably received within the sleeve.

3. The transdermal medical device of claim 1, wherein the magnetic driver element comprises a permanent magnet.

4. The transdermal medical device of claim 1, further comprising a retraction spring, the retraction spring being configured for exerting a spring force onto the movable component, the spring force being configured for moving the movable component from the proximal position into the distal position.

5. The transdermal medical device of claim 1, wherein the stationary component comprises a stop defining at least one of the distal position or the proximal position.

6. The transdermal medical device of claim 1, wherein the transdermal medical device is configured for sampling of a bodily fluid, and wherein the transdermal medical device further comprises a sampling element, wherein the sampling element is configured for receiving a sample of the bodily fluid from an incision in the skin of the user created by the penetration element.

7. The transdermal medical device of claim 1, wherein the transdermal medical device is configured for injecting a medically active component into body tissue of the user.

8. The transdermal medical device of claim 1, wherein the transdermal medical device, on the application side, comprises a device engagement element for engaging with a body mount engagement element of a body mount attached to a body surface of the user.

9. A body mount for attaching a transdermal medical device to a body surface of a user, wherein:
the transdermal medical device comprises a stationary component and a movable component, wherein:
the movable component is mounted to the stationary component in a movable manner relative to the stationary component,
the movable component is movable from a distal position to a proximal position, wherein the stationary component comprises an application side,
the movable component comprises a penetration element configured for penetrating skin of the user, wherein the penetration element, in the distal position of the movable component, is received within the stationary component and wherein the penetration element, in the proximal position of the movable component, protrudes from the application side of the stationary component, and
the movable component further comprises a magnetic driver element, wherein the magnetic driver element is configured such that a movement of the movable component is drivable by an external magnetic force; and
the body mount comprises:
an application surface,
on a side opposing the application surface, a body mount engagement element, and
a magnetic trigger element, the magnetic trigger element being configured for exerting the external magnetic force to the transdermal medical device in order to magnetically attract the magnetic driver element of the transdermal medical device when the transdermal medical device is attached to the body mount.

10. The body mount of claim 9, further comprising an adhesive element for attaching the application surface to the body surface of the user.

11. The body mount of claim 9, wherein the magnetic trigger element comprises a permanent magnet.

12. A transdermal medical kit comprising:
a transdermal medical device comprising a stationary component and a movable component, wherein:
the movable component is mounted to the stationary component in a movable manner relative to the stationary component,
the movable component is movable from a distal position to a proximal position, wherein the stationary component comprises an application side,
the movable component comprises a penetration element configured for penetrating skin of a user, wherein the penetration element, in the distal position of the movable component, is received within the stationary component and wherein the penetration element, in the proximal position of the movable component, protrudes from the application side of the stationary component, and
the movable component further comprises a magnetic driver element, wherein the magnetic driver element is configured such that a movement of the movable component is drivable by an external magnetic force; and
a body mount for attaching the transdermal medical device to a body surface of the user, wherein:
the body mount comprises an application surface for application to the body surface,
the body mount further comprises, on a side opposing the application surface, a body mount engagement element for engaging with the transdermal medical device, and
the body mount further comprises a magnetic trigger element, the magnetic trigger element being configured for exerting an external magnetic force to the transdermal medical device in order to magnetically attract the magnetic driver element of the transdermal medical device when the transdermal medical device is attached to the body mount.

13. The transdermal medical kit of claim 12, wherein:
the transdermal medical device, on the application side, comprises a device engagement element, and
the device engagement element and the body mount engagement element are configured to interact such that the transdermal medical device is aligned when the transdermal medical device is attached to the body mount.

14. The transdermal medical kit of claim 12, wherein the magnetic trigger element and the magnetic driver element are configured such that a penetration movement of the movable component is triggered by the magnetic trigger element when the body mount engagement element fully engages with the transdermal medical device.

15. A method of driving a penetration element, the method comprising:
providing a transdermal medical device, the transdermal medical device comprising a stationary component and a movable component, wherein:
the movable component is mounted to the stationary component in a movable manner relative to the stationary component,
the movable component is movable from a distal position to a proximal position, wherein the stationary component comprises an application side,
the movable component comprises the penetration element configured for penetrating skin of a user, wherein the penetration element, in the distal position of the movable component, is received within the stationary component and wherein the penetration element, in the proximal position of the movable component, protrudes from the application side of the stationary component, and
the movable component further comprises a magnetic driver element, wherein the magnetic driver element is configured such that a movement of the movable component is drivable by an external magnetic force;
providing a body mount configured for attaching the transdermal medical device to a body surface of the user, wherein:
the body mount comprises an application surface for application to the body surface,
the body mount further comprises, on a side opposing the application surface, a body mount engagement element for engaging with the transdermal medical device, and
the body mount further comprises a magnetic trigger element;
attaching the body mount to the skin of the user; and
attaching the transdermal medical device to the body mount, wherein:
the transdermal medical device engages with the body mount engagement element, and
the magnetic trigger element of the body mount exerts an external magnetic force to the transdermal medical device, thereby magnetically attracting the magnetic driver element of the transdermal medical device.
